(19) United States
(12) Patent Application Publication
(10) Pub. No.: US 2025/0022564 A1
(43) Pub. Date: Jan. 16, 2025

(54) METHOD FOR EVALUATING APPROPRIATENESS OF DOSAGE OF TARGET DRUG ADMINISTERED TO PATIENT

(71) Applicant: KAOHSIUNG MEDICAL UNIVERSITY, Kaohsiung (TW)

(72) Inventors: Chung-Hwan CHEN, Kaohsiung City (TW); Chun-Wang WEI, Kaohsiung City (TW); Pei-Cing HUANG, Kaohsiung City (TW); Hsiu-Mei CHANG, Kaohsiung City (TW); Sung-Yen LIN, Kaohsiung (TW)

(21) Appl. No.: 18/457,681

(22) Filed: Aug. 29, 2023

(30) Foreign Application Priority Data
Jul. 14, 2023 (TW) 112126427

(51) Int. Cl. G16H 20/10

(52) U.S. Cl. CPC G16H 20/10

(57) ABSTRACT

A method for evaluating appropriateness of dosage of a target drug administered to a patient is adapted to be implemented by a computing device that stores a dosage evaluation model. The method comprises steps of: obtaining at least one physiological parameter that is related to a physiological condition of the patient; obtaining at least one medication parameter that is related to a usage condition of the target drug by the patient; and feeding said at least one physiological parameter and said at least one medication parameter into the dosage evaluation model to obtain an evaluation result that indicates the appropriateness of the dosage of the target drug administered to the patient.

### CROSS-REFERENCE TO RELATED APPLICATION

This application claims priority to Taiwanese Invention patent application No. 112126427, filed on Jul. 14, 2023.

### FIELD

The disclosure relates to a method for evaluating appropriateness of dosage of a target drug administered to a patient.

### BACKGROUND

A dosage of a drug to be administered to a patient is usually determined by a medical professional based on his/her experience or population pharmacokinetics. However, appropriate dosages of a drug for patients may vary due to individual differences in physiologic conditions. Overdose may cause serious side effects and lead to medical dispute; underdose may result in less effective treatment, prolong the duration of treatment, and even cause issues like drug resistance. Conventionally, techniques of artificial intelligence have been used to determine a dosage of a drug to be administered to a patient.

### SUMMARY

Therefore, an object of the disclosure is to provide a method for evaluating appropriateness of dosage of a target drug administered to a patient and a method for establishing a dosage evaluation model.

According to one aspect of the disclosure, the method for evaluating appropriateness of dosage of a target drug administered to a patient is adapted to be implemented by a computing device that stores a dosage evaluation model. The method includes steps of: obtaining at least one physiological parameter that is related to a physiological condition of the patient; obtaining at least one medication parameter that is related to a usage condition of the target drug by the patient; and feeding said at least one physiological parameter and said at least one medication parameter into the dosage evaluation model to obtain an evaluation result that indicates the appropriateness of the dosage of the target drug administered to the patient.

According to another aspect of the disclosure, the method for establishing a dosage evaluation model is adapted to be implemented by a computing device that is configured to implement a plurality of candidate machine learning algorithms, and that stores a plurality of training data sets that are respectively related to a plurality of subjects. Each of the training data sets contains a plurality of characteristic parameters. The characteristic parameters include at least one physiological parameter that is related to a physiological condition of the corresponding one of the subjects, at least one medication parameter that is related to a usage condition of the target drug used by the corresponding one of the subjects, and a label that indicates an actual determination of appropriateness of the dosage of the target drug administered to the corresponding one of the subjects. The method includes steps of: obtaining a plurality of original candidate models based on the training data sets respectively according to the candidate machine learning algorithms, and a plurality of original model-evaluation values respectively related to the original candidate models by using a validation method; and selecting one of the original candidate models as the dosage evaluation model based on the original model-evaluation values.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating a computing device configured to implement a method for evaluating appropriateness of dosage of a target drug administered to a patient according to an embodiment of the disclosure.

FIGS. 2 to 5 are flow charts cooperatively illustrating a method for evaluating appropriateness of dosage of a target drug administered to a patient according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

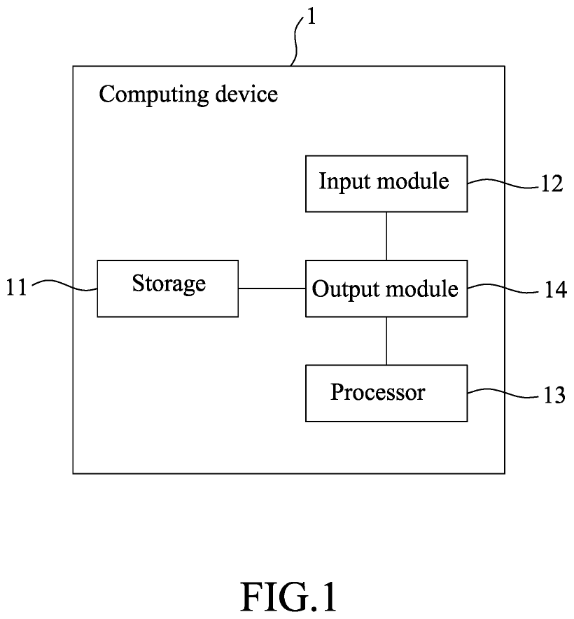

FIG. 1 illustrates an embodiment of a computing device 1 adapted to implement a method for evaluating appropriateness of dosage of a target drug administered to a patient according to the disclosure. The computing device 1 may be implemented by a desktop computer, a laptop computer, a notebook computer or a tablet computer, but is not limited thereto. The computing device 1 includes a storage 11, an input module 12, an output module 13, and a processor 14 that is electrically connected to the storage 11, the input module 12 and the output module 13.

The storage 11 may be implemented by random access memory (RAM), double data rate synchronous dynamic random access memory (DDR SDRAM), read only memory (ROM), programmable ROM (PROM), flash memory, a hard disk drive (HDD), a solid state disk (SSD), electrically-erasable programmable read-only memory (EEPROM) or any other volatile/non-volatile memory devices, but is not limited thereto.

The storage 11 is configured to store a dosage evaluation model, a plurality of to-be-processed data sets that are respectively related to a plurality of subjects (e.g., people who have been treated with the target drug in the past), and a plurality of training data sets that are related to some or all of the subjects. It is worth to note that the to-be-processed data sets are derived from clinical data under the regulations of the Institutional Review Board (IRB) for ethics in human subject research, and the training data sets are obtained from the to-be-processed data sets. The clinical data at least includes main files of inpatient applications, detail files of inpatient applications, files of inspection reports, files of bacteriological examination reports, files of personal information of patients, files of medical advices for inpatients, and files of physiological examinations.

Each of the to-be-processed data sets is supposed to contain a plurality of characteristic parameters. The characteristic parameters include at least one physiological parameter that is related to a physiological condition of the corresponding one of the subjects (hereinafter referred to as “the corresponding subject” for simplicity), at least one medication parameter that is related to a usage condition of the target drug by the corresponding subject, and a label that indicates an actual determination of appropriateness of the dosage of the target drug administered to the corresponding subject. In a first embodiment of the method where the target drug is vancomycin, said at least one physiological parameter includes a gender of the corresponding subject, a blood urea nitrogen (BUN) level of the corresponding subject, a creatinine level of the corresponding subject, a body mass index (BMI) of the corresponding subject, and an age of the corresponding subject. Said at least one medication parameter includes a fixed dosage of the target drug administered to the corresponding subject every time, a frequency of administering the fixed dosage of the target drug to the corresponding subject, a treatment duration of using the target drug by the corresponding subject, and an observation time interval from a final-administration time when the target drug was last administered to the corresponding subject at the end of the treatment duration to an examination time when a concentration of the target drug in the blood of the corresponding subject was measured. It is worth to note that the observation time interval can be counted in days. The label exemplarily indicates one of a plurality of dosage levels, such as an underdose level, an overdose level, a medium-dose level between the underdose level and the overdose level, a low-dose level between the medium-dose level and the underdose level, and a high-dose level between the medium-dose level and the overdose level. Specifically, the medium-dose level indicates that the dosage of the target drug is appropriate. In this embodiment, a concentration of the target drug (i.e., vancomycin) in the blood of less than 15 mg/dl would be considered the underdose level; a concentration of the target drug in the blood of not less than 15 mg/dl but less than 16.7 mg/dl would be considered the low-dose level; a concentration of the target drug in the blood of not less than 16.7 mg/dl but less than 18.4 mg/dl would be considered the medium-dose level; a concentration of the target drug in the blood of not less than 18.4 mg/dl but less than 20 mg/dl would be considered the high-dose level; and a concentration of the target drug in the blood of not less than 20 mg/dl would be considered the overdose level. In one embodiment, the label is one of the underdose level, the medium-dose level and the overdose level (i.e., there is no high-dose level or low-dose level). In one embodiment, the label is one of an appropriate-dose level and an inappropriate-dose level. In one embodiment, the label is a numerical value that represents a dose level (e.g., “1” representing the underdose level, “2” representing the low-dose level; “3” representing the medium-dose level; “4” representing the high-dose level and “5” representing the overdose level) or a concentration of the target drug in the blood.

Each of the training data sets contains a plurality of characteristic parameters. The characteristic parameters include at least one physiological parameter that is related to a physiological condition of the corresponding subject, at least one medication parameter that is related to a usage condition of the target drug by the corresponding subject, and a label that indicates an actual determination of appropriateness of the dosage of the target drug administered to the corresponding subject.

The input module 12 may be implemented by a keyboard, or may be implemented to be a network interface controller or a wireless transceiver that supports wireless communication standards, such as Bluetooth® technology standards, Wi-Fi technology standards and/or cellular network technology standards, but is not limited thereto. In embodiments where the input module 12 is implemented by a keyboard, the to-be-processed data sets may be manually typed into the computing device 1 for storage by using the input module 12; in embodiments where the input module 12 is implemented by a network interface controller or a wireless transceiver, the to-be-processed data sets may be obtained by the computing device 1 from a remote database (not shown) that is accessible by the input module 12.

The output module 13 may be a liquid-crystal display (LCD), a light-emitting diode (LED) display, a plasma display panel, a projection display or the like. However, implementation of the output module 13 is not limited to the disclosure herein and may vary in other embodiments.

The processor 14 may be implemented by a central processing unit (CPU), a microprocessor, a micro control unit (MCU), a system on a chip (SoC), or any circuit configurable/programmable in a software manner and/or hardware manner to implement functionalities discussed in this disclosure. The processor 14 is configured to implement a plurality of candidate machine learning algorithms, such as algorithms related to a Bayesian network (also known as Bayes net), sequential minimal optimization (SMO), bootstrap aggregating (also known as bagging), a J48 decision tree classification algorithm, and a random forest algorithm.

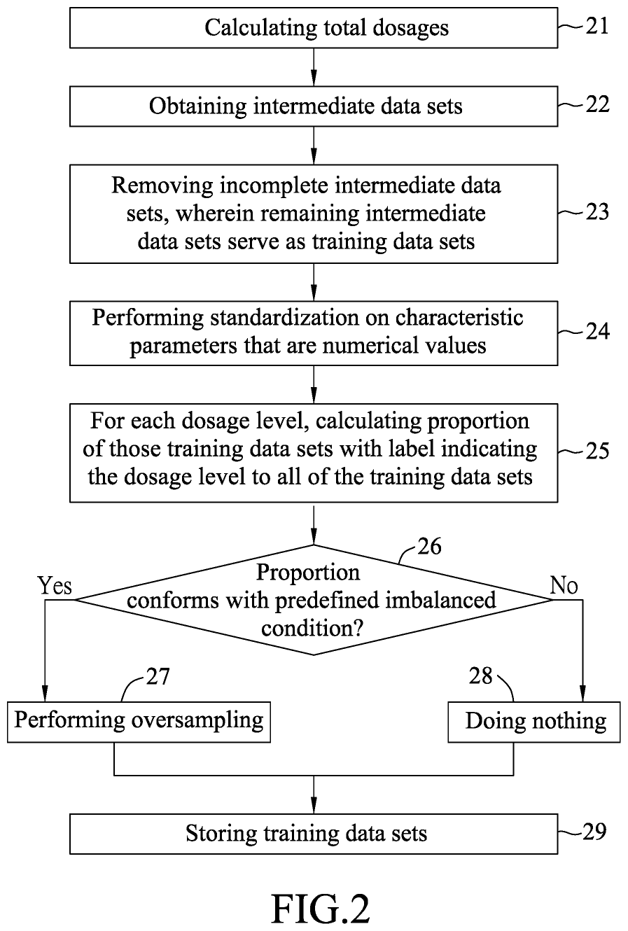

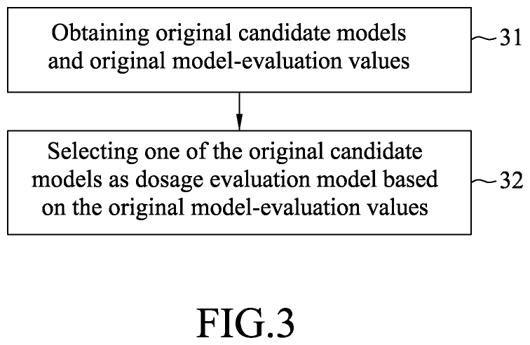

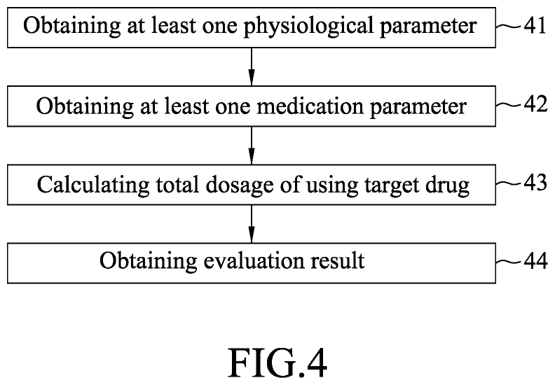

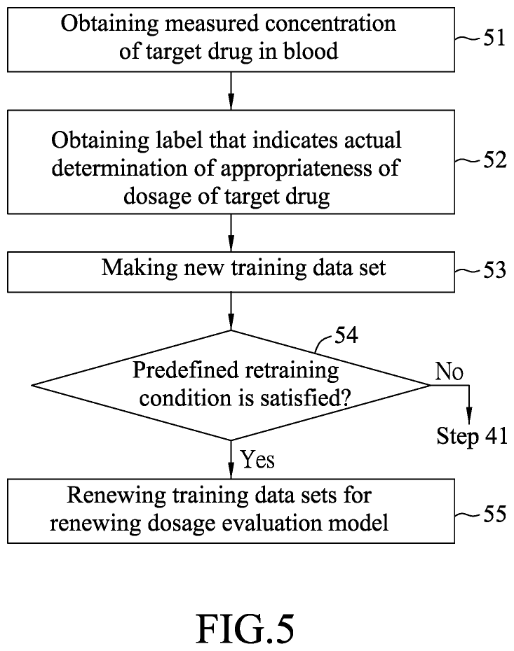

Referring to FIGS. 2 to 5, an embodiment of the method for evaluating appropriateness of dosage of a target drug administered to a patient according to the disclosure is illustrated. The method includes a data processing procedure as shown in FIG. 2, a model establishing procedure as shown in FIG. 3, an evaluating procedure as shown in FIG. 4, and a feedback procedure as shown in FIG. 5.

Referring to FIG. 2, the data processing procedure is for obtaining the training data sets, and includes steps 21 to 29 as delineated below.

In step 21, for each of the to-be-processed data sets, the processor 14 calculates a total dosage based on the fixed dosage of the target drug administered to the corresponding subject each time, the frequency of administering the fixed dosage of the target drug to the corresponding subject, and the treatment duration of using the target drug by the corresponding subject.

In step 22, the processor 14 obtains a plurality of intermediate data sets respectively based on the to-be-processed data sets. Specifically, each of the intermediate data sets includes at least one medication parameter that includes the observation time interval and the total dosage of the respective one of the to-be-processed data sets (hereinafter referred to as “the respective to-be-processed data set” for simplicity), at least one physiological parameter that includes said at least one physiological parameter of the respective to-be-processed data set, and the label of the respective to-be-processed data set.

In step 23, for each of the intermediate data sets, the processor 14 determines whether the intermediate data set is missing any one of the characteristic parameters that are supposed to be present, and when it is determined that the intermediate data set is missing any one of the characteristic parameters, the processor 14 deletes the intermediate data set. Those of the intermediate data sets that remain serve as the training data sets. In other words, each of the to-be-processed data sets is supposed to contain a predetermined number of characteristic parameters (e.g., ten characteristic parameters including gender, BUN level, creatinine level, BMI, age, fixed dosage, frequency of taking the target drug, treatment duration, observation time interval, and label); when one of the to-be-processed data sets does not contain all of these characteristic parameters, the intermediate data set that is derived from said to-be-processed data set cannot serve as a training data set, and is discarded in step 23.

In step 24, for each of the training data sets, the processor 14 performs standardization (e.g., z-score normalization) on any of the characteristic parameters of the training data set that is a numerical value. For example, the processor 14 normalizes a numerical characteristic parameter of each of the training data sets with population parameters (e.g., mean and standard deviation) derived from corresponding numerical characteristic parameters respectively of the training data sets.

In step 25, for each of the dosage levels (e.g., the underdose level, the low-dose level, the medium-dose level, the high-dose level, and the overdose level), the processor 14 calculates a proportion of those of the training data sets that each contain the label indicating the dosage level to all of the training data sets.

The flow of procedure then proceeds to steps 26 to 28, which are performed with respect to each of the dosage levels. In step 26, the processor 14 determines whether the proportion for the dosage level conforms with a predefined imbalanced condition. The predetermined imbalanced condition is exemplarily that the proportion is not greater than a threshold value. When it is determined that the proportion conforms with the predefined imbalanced condition, step 27 is performed. On the other hand, when it is determined that the proportion does not conform with the predefined imbalanced condition, step 28 is performed.

In step 27, the processor 14 performs oversampling on those of the training data sets that each contain the label indicating the dosage level. In particular, the processor 14 uses synthetic minority over-sampling technique (SMOTE) in step 27.

In step 28, the processor 14 leaves those of the training data sets that each contain the label indicating the dosage level as is.

It should be noted herein that the design of steps 26 to 28 is to balance out any potential imbalanced nature of the original training data sets. In this way, the resulting training data sets may be relatively more balanced, thereby improving accuracy of evaluation made by the dosage evaluation model that is to be trained using the training data sets.

In step 29, the processor 14 stores the training data sets that have been processed (for balance of data) in the storage 11.

Referring to FIG. 3, the model establishing procedure is for establishing the dosage evaluation model used to evaluate appropriateness of dosage of the target drug administered to a patient, and includes steps 31 and 32 delineated below.

In step 31, the processor 14 obtains a plurality of original candidate models based on the training data sets respectively according to the candidate machine learning algorithms, and a plurality of original model-evaluation values respectively related to the original candidate models by using a validation method. The validation method is exemplarily 10-fold cross-validation, but is not limited thereto.

In step 32, the processor 14 selects one of the original candidate models as the dosage evaluation model based on the original model-evaluation values. Particularly, the processor 14 selects one of the original candidate models that corresponds to a greatest one of the original model-evaluation values as the dosage evaluation model.

Referring to FIG. 4, the evaluating procedure is for evaluating appropriateness of dosage of the target drug administered to a patient, and includes steps 41 to 44 delineated below.

In step 41, the processor 14 obtains from the input module 12 at least one physiological parameter that is related to a physiological condition of the patient. For the first embodiment of the method where the target drug is vancomycin, said at least one physiological parameter includes a gender of the patient, a BUN level of the patient, a creatinine level of the patient, a BMI of the patient, and an age of the patient. It is worth to note that said at least one physiological parameter may be obtained from a remote database in a hospital where the patient has visited in the past.

In step 42, the processor 14 obtains from the input module 12 at least one medication parameter that is related to a usage condition of the target drug by the patient. Said at least one medication parameter includes a fixed dosage of the target drug administered to the patient each time during a current treatment, a frequency of administering the fixed dosage of the target drug to the patient during the current treatment, and a duration of the current treatment. It is worth to note that said at least one medication parameter may also be obtained from the aforesaid remote database in the hospital where the patient has visited in the past.

In step 43, the processor 14 calculates a total dosage of the target drug used by the patient as an additional medication parameter based on the fixed dosage of the target drug administered to the patient each time during the current treatment, the frequency of administering the fixed dosage of the target drug to the patient during the current treatment, and the duration of the current treatment. It is worth to note that in some embodiments, the total dosage is manually inputted into the computing device 1 by using the input module 12, and step 43 is omitted.

In step 44, the processor 14 feeds said at least one physiological parameter and said at least one medication parameter into the dosage evaluation model to obtain an evaluation result that indicates the appropriateness of the dosage of the target drug administered to the patient. For the first embodiment where the target drug is vancomycin, the gender of the patient, the BUN level of the patient, the creatinine level of the patient, the BMI of the patient, the age of the patient, the total dosage of the target drug administered to the patient during the current treatment, and the observation time interval relating to the target drug for the patient are fed into the dosage evaluation model to obtain the evaluation result. In this embodiment, the evaluation result is one of the underdose level, the overdose level, the medium-dose level, the low-dose level, and the high-dose level. In one embodiment, the evaluation result may be one of the underdose level, the medium-dose level and the overdose level. In one embodiment, the evaluation result may be the appropriate-dose level or the inappropriate-dose level. In one embodiment, the evaluation result is a numerical value that represents an estimated dose level (e.g., “1” representing the underdose level, “2” representing the low-dose level; “3” representing the medium-dose level; “4” representing the high-dose level; and “5” representing the overdose level) or a concentration of the target drug in the blood of the patient.

Thereafter, the processor 14 outputs the evaluation result to the output module 13 for presenting the same.

Referring to FIG. 5, the feedback procedure is for retraining the dosage evaluation model, and includes steps 51 to 55 delineated below.

In step 51, the processor 14 obtains from the input module 12 a measured concentration of the target drug in the blood of the patient after the target drug was administered to the patient. It is worth to note that the measured concentration may also be obtained from the aforesaid remote database in the hospital where the patient has visited in the past.

In step 52, the processor 14 obtains a label that indicates an actual determination of appropriateness of the dosage of the target drug administered to the patient that is made based on the measured concentration thus obtained. For the first embodiment where the target drug is vancomycin, the label is one of the underdose level, the low-dose level, the medium-dose level, the high-dose level and the overdose level.

In step 53, the processor 14 makes a new training data set that contains said at least one physiological parameter related to the physiological condition of the patient, said at least one medication parameter related to the usage condition of the target drug by the patient, and the label thus obtained. It should be noted that said at least one medication parameter further includes an observation time interval from a final-administration time when the target drug was last administered to the patient at the end of the current treatment (see step 41) to an examination time when the measured concentration of the target drug in blood of the patient was measured after termination of the current treatment.

In step 54, the processor 14 determines whether a predefined retraining condition is satisfied. When it is determined that the predefined retraining condition is satisfied, the flow of procedure proceeds to step 55. Otherwise, when it is determined that the predefined retraining condition is not satisfied, the flow of procedure goes back to step 41 so as to make evaluation for another patient. In some embodiments, the method is terminated when it is determined that the predefined retraining condition is not satisfied. In one embodiment, the predefined retraining condition is that a total number of new training data sets that have been made in step 53 is greater than a predetermined threshold number (e.g., 300). In one embodiment, the predefined retraining condition is that the training data sets have not been renewed for a predetermined time period (e.g., one month).

In step 55, the processor 14 renews the training data sets by adding into the training data sets the new training data set(s) made in step 53. Then, the processor 14 obtains a plurality of new candidate models based on the training data sets thus renewed respectively according to the candidate machine learning algorithms, and a plurality of new model-evaluation values respectively related to the new candidate models by using the validation method. Moreover, the processor 14 selects one of the new candidate models as the dosage evaluation model based on the new model-evaluation values. Particularly, the processor 14 selects one of the new candidate models that corresponds to a greatest one of the new model-evaluation values as the dosage evaluation model. In this way, the dosage evaluation model may be refined.

Table 1 below provides an example of a confusion matrix related to evaluation results obtained using the dosage evaluation model respectively for 299 patients.

TABLE 1

| | | Evaluation results | | | | | |
|---|---|---|---|---|---|---|---|
| | | Under- | Low- | Medium- | High- | Over- | |
| | | dose | dose | dose | dose | dose | Total |
| Actual | Underdose | 134 | 0 | 0 | 2 | 1 | 137 |
| results | Low-dose | 11 | 12 | 1 | 1 | 2 | 27 |
| | Medium- | 6 | 2 | 23 | 0 | 3 | 34 |
| | dose | | | | | | |
| | High-dose | 1 | 0 | 1 | 16 | 6 | 24 |
| | Overdose | 2 | 2 | 4 | 1 | 68 | 77 |
| | Total | 154 | 16 | 29 | 20 | 80 | 299 |

According to Table 1, for the evaluation results made by using the dosage evaluation model, a true positive rate (TPR) for the underdose level is 134/137=97.81%, a TPR for the low-dose level is 12/27=44.44%, a TPR for the medium-dose level is 23/34=67.64%, a TPR for the high-dose level is 16/24=66.66%, a TPR for the overdose level is 68/77=88.31%, a true negative rate (TNR) for the underdose level is 119/162=73.45%, a TNR for the low-dose level is 241/272=88.6%, a TNR for the medium-dose level is 230/265=86.79%, a TNR for the high-dose level is 237/275=86.18%, and a TNR for the overdose level is 185/222=83.33%. Further, for the evaluation results made by using the dosage evaluation model, the precision for the underdose level is 134/154=87.01%, the precision for the low-dose level is 12/16=75%, the precision for the medium-dose level is 23/29=79.31%, the precision for the high-dose level is 16/20=80%, and the precision for the overdose level is 68/80=85%. Furthermore, for the evaluation results made by using the dosage evaluation model, the recall for the underdose level is 134/137=97.81%, the recall for the low-dose level is 12/27=44.44%, the recall for the medium-dose level is 23/34=67.64%, the recall for the high-dose level is 16/24=66.66%, and the recall for the overdose level is 68/77=88.31%.

In clinical practice, an amount of vancomycin administered to a patient would gradually increase during the treatment duration. However, because of a high risk of serious side effects caused by using vancomycin, medical professionals usually tend to be conservative on the amount of vancomycin to be administered. It is worth to note that according Table 1, a number of patients using an underdose level of vancomycin occupy 45.81% of all patients (i.e., 137/299=45.81%). Additionally, the TPR for the overdose level shows that the probability of using the dosage evaluation model to correctly evaluate a dosage regimen as an overdose level is 88.31%. Therefore, inappropriately delivering an overdose level of vancomycin to a specific patient may be prevented by using the dosage evaluation model to evaluate the appropriateness of dosage of vancomycin administered to the specific patient according to this disclosure, and the risk of serious side effects caused by using vancomycin may thereby be reduced. The aforesaid value of recall for the underdose level shows that a probability of using the dosage evaluation model to correctly evaluate a dosage regimen as an underdose level is 97.81% (true positive rate (TPR) for the underdose level is 97.81%). Thus, inappropriately delivering an underdose level of vancomycin to a patient may be prevented by using the dosage evaluation mode, thereby improving effectiveness of treatment and alleviating suffering of the patient.

In a second embodiment of the method, the target drug is lidocaine. Since the second embodiment is similar to the first embodiment, only differences between the first embodiment and the second embodiment will be explained in the following paragraphs for the sake of brevity.

For each of the to-be-processed data sets, said at least one physiological parameter includes a gender of the corresponding subject, a date of visit that the corresponding subject had a clinic visit, a date of admission when the corresponding subject was admitted to be hospitalized as an inpatient, a smoking/non-smoking habit of the corresponding subject, an alcohol-use habit of the corresponding subject, at least one type of long-term medication used by the corresponding subject, a chronic condition of the corresponding subject, a creatinine level of the corresponding subject, a creatinine clearance level of the corresponding subject, an estimated glomerular filtration rate (eGFR) of the corresponding subject, an alkaline phosphatase (ALP) level of the corresponding subject, an albumin level of the corresponding subject, a BMI of the corresponding subject, and an age of the corresponding subject; said at least one medication parameter includes a historic medication usage record of the corresponding subject with respect to the target drug, a fixed dosage of the target drug administered to the corresponding subject each time during a recent treatment, a frequency of administering the fixed dosage of the target drug to the corresponding subject during the recent treatment, a duration (which is counted in days) of the recent treatment, an observation time interval from a final-administration time when the target drug was last administered to the corresponding subject at the end of the recent treatment to an examination time when a concentration of the target drug in the blood of the corresponding subject was measured in an examination performed on the corresponding subject after termination of the recent treatment, a date of the examination, the examination time, a trough level (i.e., the lowest level) of the concentration of the target drug in the blood of the corresponding subject as measured in the examination, and a peak (i.e., the highest level) of the concentration of the target drug in the blood of the corresponding subject as measured in the examination, wherein the historic medication usage record includes dates, a number of days and a frequency of a previous treatment of administering the target drug to the corresponding subject, and a total dosage of the target drug administered to the corresponding subject in the previous treatment. A concentration of the target drug (i.e., lidocaine) in the blood of less than 1.5 mg/dl would be considered the underdose level; a concentration of the target drug in the blood of not less than 1.5 mg/dl but less than 3 mg/dl would be considered the low-dose level; a concentration of the target drug in the blood of not less than 3 mg/dl but less than 4.5 mg/dl would be considered the medium-dose level; a concentration of the target drug in the blood of not less than 4.5 mg/dl but less than 6 mg/dL would be considered the high-dose level; and a concentration of the target drug in the blood of not less than 6 mg/dl would be considered the overdose level.

For each of the training data sets obtained by way performing steps 22 and 23 based on the to-be-processed data sets, said at least one medication parameter of the training data set includes the total dosage of the target drug in the recent treatment, the historic medication usage record (i.e., the date, time, and total dosage relating to the previous treatment), the date of examination, the examination time, the trough level as measured in the examination, the peak as measured in the examination, and the observation time interval of the corresponding one of the to-be-processed data sets.

Said at least one physiological parameter obtained by the processor 14 in step 41 includes a gender of the patient, a date of visit that the patient had a clinic visit, a date of admission that the patient was admitted as an inpatient, a smoking/non-smoking habit of the patient, an alcohol-use habit of the patient, at least one type of long-term medication used by the patient, a chronic condition of the patient, a creatinine level of the patient, a creatinine clearance level of the patient, an eGFR of the patient, an ALP level of the patient, an albumin level of the patient, a BMI of the patient, and an age of the patient.

Said at least one medication parameter obtained by the processor 14 in step 41 includes a historic medication usage record of the patient with respect to the target drug, a fixed dosage of the target drug administered to the patient each time during a current treatment, a frequency of administering the fixed dosage of the target drug to the patient during the current treatment, a duration (which is counted in days) of the current treatment, an observation time interval from a final-administration time when the target drug was last administered to the patient at the end of the current treatment to an examination time when a concentration of the target drug in the blood of the patient was measured in an examination performed on the patient after termination of the current treatment, a date of the examination, the examination time, a trough level (i.e., the lowest level) of the concentration of the target drug in the blood of the patient as measured in the examination, and a peak (i.e., the highest level) of the concentration of the target drug in the blood of the patient as measured in the examination, wherein the historic medication usage record includes date and time of a previous treatment of administering the target drug to the patient, and a total dosage of the target drug administered to the patient in the previous treatment.

The gender of the patient, the date of examination for the patient, the date of admission for the patient, the smoking/non-smoking habit of the patient, the alcohol-use habit of the patient, said at least one type of long-term medication used by the patient, the chronic condition of the patient, the creatinine level of the patient, the creatinine clearance level of the patient, the eGFR of the patient, the ALP level of the patient, the albumin level of the patient, the BMI of the patient, the age of the patient, the total dosage in the current treatment for the patient, the observation time interval for the patient, the historic medication usage record (i.e., the date, time and total dosage relating to the previous treatment) for the patient, the date of the examination for the patient, the examination time for the patient, the trough level as measured in the examination for the patient, and the peak as measured in the examination for the patient are fed into the dosage evaluation model to obtain the evaluation result.

In a third embodiment of the method, the target drug is digoxin which is used to treat congestive heart failure. Since the third embodiment is similar to the first embodiment, only differences between the first embodiment and the third embodiment will be explained in the following paragraphs for the sake of brevity.

For each of the to-be-processed data sets, said at least one physiological parameter includes a gender of the corresponding subject, a smoking/non-smoking habit of the corresponding subject, an alcohol-use habit of the corresponding subject, at least one type of long-term medication used by the corresponding subject, a chronic condition of the corresponding subject, a creatinine level of the corresponding subject, an eGFR of the corresponding subject, an ALP level of the corresponding subject, a BMI of the corresponding subject, and an age of the corresponding subject; said at least one medication parameter includes a historic medication usage record of the corresponding subject with respect to the target drug, a fixed dosage of the target drug administered to the corresponding subject each time during a recent treatment, a frequency of administering the fixed dosage of the target drug to the corresponding subject during the recent treatment, a duration (which is counted in days) of the recent treatment, an observation time interval from a final-administration time when the target drug was last administered to the corresponding subject at the end of the recent treatment to an examination time when a concentration of the target drug in the blood of the corresponding subject was measured in an examination performed on the corresponding subject after termination of the recent treatment, a date of the examination, the examination time, and a trough level (i.e., the lowest level) of the concentration of the target drug in the blood of the corresponding subject as measured in the examination, wherein the historic medication usage record includes date and time of a previous treatment of administering the target drug to the corresponding subject, and a total dosage of the target drug administered to the corresponding subject in the previous treatment. A concentration of the target drug (i.e., digoxin) in the blood of less than 0.8 mg/dl would be considered the underdose level; a concentration of the target drug in the blood of not less than 0.8 mg/dl but less than 1.2 mg/dl would be considered the low-dose level; a concentration of the target drug in the blood of not less than 1.2 mg/dl but less than 1.6 mg/dl would be considered the medium-dose level; a concentration of the target drug in the blood of not less than 1.6 mg/dl but less than 2 mg/dl would be considered the high-dose level; and a concentration of the target drug in the blood of not less than 2 mg/dl would be considered the overdose level.

For each of the training data sets obtained by way of performing steps 22 and 23, said at least one medication parameter of the training data set includes the total dosage of the target drug, the historic medication usage record (i.e., the total dosage, date, and time relating to the previous treatment), the date of the examination, the examination time, the trough level as measured in the examination, and the observation time interval of the corresponding one of the to-be-processed data sets.

Said at least one physiological parameter obtained by the processor 14 in step 41 includes a gender of the patient, a smoking/non-smoking habit of the patient, an alcohol-use habit of the patient, at least one type of long-term medication used by the patient, a chronic condition of the patient, a creatinine level of the patient, an eGFR of the patient, an ALP level of the patient, a BMI of the patient, and an age of the patient.

Said at least one medication parameter obtained by the processor 14 in step 41 includes a historic medication usage record of the patient with respect to the target drug, a fixed dosage of the target drug administered to the patient each time during a current treatment, a frequency of administering the fixed dosage of the target drug to the patient during the current treatment, a duration (which is counted in days) of the current treatment, an observation time interval from a final-administration time when the target drug was last administered to the patient at the end of the current treatment to an examination time when a concentration of the target drug in the blood of the patient was measured in an examination performed on the patient after termination of the current treatment, a date of the examination, the examination time, and a trough level (i.e., the lowest level) of the concentration of the target drug in the blood of the patient as measured in the examination, wherein the historic medication usage record includes date and time of a previous treatment of administering the target drug to the patient, and a total dosage of the target drug administered to the patient in the previous treatment.

The gender of the patient, the smoking/non-smoking habit of the patient, the alcohol-use habit of the patient, said at least one type of long-term medication used by the patient, the chronic condition of the patient, the creatinine level of the patient, the eGFR of the patient, the ALP level of the patient, the BMI of the patient, the age of the patient, the total dosage in the current treatment for the patient, the observation time interval for the patient, the historic medication usage record (i.e., the date, time and total dosage in the previous treatment) for the patient, the date of the examination for the patient, the examination time for the patient, and the trough level as measured in the examination for the patient are fed into the dosage evaluation model to obtain the evaluation result.

In a fourth embodiment of the method, the target drug is digoxin, which is used to treat atrial arrhythmia in this embodiment. Since the fourth embodiment is similar to the third embodiment, only differences between the third embodiment and the fourth embodiment will be explained in the following paragraphs for the sake of brevity.

A concentration of the target drug (i.e., digoxin) in the blood of less than 1.5 mg/dl would be considered the underdose level; a concentration of the target drug in the blood of not less than 1.5 mg/dl but less than 1.8 mg/dl would be considered the low-dose level; a concentration of the target drug in the blood of not less than 1.8 mg/dl but less than 2.2 mg/dL would be considered the medium-dose level; a concentration of the target drug in the blood of not less than 2.2 mg/dl but less than 2.5 mg/dl would be considered the high-dose level; and a concentration of the target drug in the blood of not less than 2.5 mg/dL would be considered the overdose level.

To sum up, in the method according to the disclosure, a dosage evaluation model is used to obtain an evaluation result that indicates the appropriateness of the dosage of the target drug administered to the patient based on at least one physiological parameter that is related to a physiological condition of the patient and based on at least one medication parameter that is related to a usage condition of the target drug by the patient. Moreover, the dosage evaluation model can be further refined by retraining the dosage evaluation model using training data sets that are renewed.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for evaluating appropriateness of dosage of a target drug administered to a patient, adapted to be implemented by a computing device that stores a dosage evaluation model, the method comprising steps of:
obtaining at least one physiological parameter that is related to a physiological condition of the patient;
obtaining at least one medication parameter that is related to a usage condition of the target drug by the patient; and
feeding said at least one physiological parameter and said at least one medication parameter into the dosage evaluation model to obtain an evaluation result that indicates the appropriateness of the dosage of the target drug administered to the patient.

2. The method as claimed in claim 1, the computing device being configured to implement a plurality of candidate machine learning algorithms, and further storing a plurality of training data sets that are respectively related to a plurality of subjects, each of the training data sets containing a plurality of characteristic parameters, the characteristic parameters including at least one physiological parameter that is related to a physiological condition of the corresponding one of the subjects, at least one medication parameter that is related to a usage condition of the target drug by the corresponding one of the subjects, and a label that indicates an actual determination of appropriateness of the dosage of the target drug administered to the corresponding one of the subjects, the method further comprising steps of:
obtaining a plurality of original candidate models based on the training data sets respectively according to the candidate machine learning algorithms, and a plurality of original model-evaluation values respectively related to the original candidate models by using a validation method; and
selecting one of the original candidate models as the dosage evaluation model based on the original model-evaluation values.

3. The method as claimed in claim 2, further comprising, after the step of feeding said at least one physiological parameter and said at least one medication parameter into the dosage evaluation model to obtain the evaluation result, steps of:
obtaining a measured concentration of the target drug in blood of the patient after the target drug is administered to the patient;
obtaining a label that indicates an actual determination of appropriateness of the dosage of the target drug administered to the patient based on the measured concentration thus obtained;
determining whether a predefined retraining condition is satisfied; and
when it is determined that the predefined retraining condition is satisfied,
renewing the training data sets by adding into the training data sets a new training data set that contains said at least one physiological parameter related to the physiological condition of the patient, said at least one medication parameter related to the usage condition of the target drug by the patient, and the label thus obtained,
obtaining a plurality of new candidate models based on the training data sets thus renewed respectively according to the candidate machine learning algorithms, and a plurality of new model-evaluation values respectively related to the new candidate models by using the validation method, and
selecting one of the new candidate models as the dosage evaluation model based on the new model-evaluation values.

4. The method as claimed in claim 3, wherein the predefined retraining condition is that a total number of new training data sets to be added into the training data sets is greater than a predetermined threshold number.

5. The method as claimed in claim 3, wherein the predefined retraining condition is that the training data sets have not been renewed for a predetermined time period.

6. The method as claimed in claim 2, wherein the label indicates one of a plurality of dosage levels, the method further comprising, before the step of obtaining a plurality of original candidate models and a plurality of original model-evaluation values, steps, to be performed with respect to each of the dosage levels, of:
calculating a proportion of those of the training data sets that each contain the label indicating the dosage level to all of the training data sets;
determining whether the proportion conforms with a predefined imbalanced condition; and
when it is determined that the proportion conforms with the predefined imbalanced condition, balancing the training data sets by performing oversampling on said those of the training data sets that each contain the label indicating the dosage level,
wherein the step of obtaining a plurality of original candidate models and a plurality of original model-evaluation values is to obtain the original candidate models based on the training data sets thus balanced.

7. The method as claimed in claim 6, wherein the step of balancing the training data sets is to balance the training data sets by using synthetic minority over-sampling technique (SMOTE).

8. The method as claimed in claim 2, the computing device further storing a plurality of to-be-processed data sets that are respectively related to a plurality of subjects at least including the subjects to which the training data sets are respectively related, each of the to-be-processed data sets containing a plurality of characteristic parameters, the characteristic parameters including at least one physiological parameter that is related to a physiological condition of the corresponding one of the subjects, at least one medication parameter that is related to a usage condition of the target drug by the corresponding one of the subjects, and a label that indicates an actual determination of appropriateness of the dosage of the target drug administered to the corresponding one of the subjects, said at least one medication parameter including a fixed dosage of the target drug administered to the corresponding one of the subjects each time, a frequency of administering the fixed dosage of the target drug to the corresponding one of the subjects, a treatment duration of using the target drug by the corresponding one of the subjects, and an observation time interval from a final-administration time instant when the target drug was last administered to the corresponding one of the subjects at the end of the treatment duration to an examination time when a concentration of the target drug in blood of the corresponding one of the subjects was measured, the method further comprising, prior to the step of obtaining a plurality of original candidate models and a plurality of original model-evaluation values, steps of:
for each of the to-be-processed data sets, calculating a total dosage based on the fixed dosage of the target drug administered to the corresponding one of the subjects each time, the frequency of administering the fixed dosage of the target drug to the corresponding one of the subjects, and the treatment duration of using the target drug by the corresponding one of the subjects; and
obtaining a plurality of intermediate data sets respectively based on the to-be-processed data sets, wherein each of the intermediate data sets includes the observation time interval and the total dosage of the respective one of the to-be-processed data sets, said at least one physiological parameter of the respective one of the to-be-processed data sets, and the label of the respective one of the to-be-processed data sets,
wherein the intermediate data sets serve as the training data sets, and for each of the training data sets, the observation time interval and the total dosage of the respective one of the intermediate data sets serve as said at least one of the medication parameter of the training dataset, said at least one physiological parameter of the respective one of the intermediate data sets serves as said at least one physiological parameter of the training data set, and the label of the respective one of the intermediate data sets serves as the label of the training data set.

9. The method as claimed in claim 2, further comprising, prior to the step of obtaining a plurality of original candidate models and a plurality of original model-evaluation values, a step of:
for each of the intermediate data sets, determining whether the intermediate data set is missing any one of the characteristic parameters, and when it is determined that the intermediate data set is missing any one of the characteristic parameters, deleting the intermediate data set;
wherein those of the intermediate data sets that remain serve as the training data sets.

10. The method as claimed in claim 2, further comprising, prior to the step of obtaining a plurality of original candidate models and a plurality of original model-evaluation values, a steps, to be performed with respect to each of the training data sets, of:
performing standardization on any of the characteristic parameters of the training data set that is a continuous variable.

11. A method for establishing a dosage evaluation model used to evaluate appropriateness of dosage of a target drug administered to a patient, adapted to be implemented by a computing device that is configured to implement a plurality of candidate machine learning algorithms, and that stores a plurality of training data sets that are respectively related to a plurality of subjects, each of the training data sets containing a plurality of characteristic parameters, the characteristic parameters including at least one physiological parameter that is related to a physiological condition of the corresponding one of the subjects, at least one medication parameter that is related to a usage condition of the target drug used by the corresponding one of the subjects, and a label that indicates an actual determination of appropriateness of the dosage of the target drug administered to the corresponding one of the subjects, the method comprising steps of:
obtaining a plurality of original candidate models based on the training data sets respectively according to the candidate machine learning algorithms, and a plurality of original model-evaluation values respectively related to the original candidate models by using a validation method; and
selecting one of the original candidate models as the dosage evaluation model based on the original model-evaluation values.

12. The method as claimed in claim 11, wherein the label indicates one of a plurality of dosage levels, the method further comprising, before the step of obtaining a plurality of original candidate models and a plurality of original model-evaluation values, steps, to be performed with respect to each of the dosage levels, of:
calculating a proportion of those of the training data sets that each contain the label indicating the dosage level to all of the training data sets;
determining whether the proportion conforms with a predefined imbalanced condition; and
when it is determined that the proportion conforms with the predefined imbalanced condition, balancing the training data sets by performing oversampling on said those of the training data sets that each contain the label indicating the dosage level,
wherein the step of obtaining a plurality of original candidate models and a plurality of original model-evaluation values is to obtain the original candidate models based on the training data sets thus balanced.

13. The method as claimed in claim 12, wherein the step of balancing the training data sets is to balance the training data sets by using synthetic minority over-sampling technique (SMOTE).

14. The method as claimed in claim 11, the computing device further storing a plurality of to-be-processed data sets that are respectively related to a plurality of subjects at least including the subjects to which the training data sets are respectively related, each of the to-be-processed data sets containing a plurality of characteristic parameters, the characteristic parameters including at least one physiological parameter that is related to a physiological condition of the corresponding one of the subjects, at least one medication parameter that is related to a usage condition of the target drug by the corresponding one of the subjects, and a label that indicates an actual determination of appropriateness of the dosage of the target drug administered to the corresponding one of the subjects, said at least one medication parameter including a fixed dosage of the target drug administered to the corresponding one of the subjects each time, a frequency of administering the fixed dosage of the target drug to the corresponding one of the subjects, a treatment duration of using the target drug by the corresponding one of the subjects, and an observation time interval from a final-administration time instant when the target drug was last administered to the corresponding one of the subjects at the end of the treatment duration to an examination time when a concentration of the target drug in blood of the corresponding one of the subjects was measured, the method further comprising, prior to the step of obtaining a plurality of original candidate models and a plurality of original model-evaluation values, steps of:
for each of the to-be-processed data sets, calculating a total dosage based on the fixed dosage of the target drug administered to the corresponding one of the subjects each time, the frequency of administering the fixed dosage of the target drug to the corresponding one of the subjects, and the treatment duration of using the target drug by the corresponding one of the subjects; and
obtaining a plurality of intermediate data sets respectively based on the to-be-processed data sets, wherein each of the intermediate data sets includes the observation time interval and the total dosage of the respective one of the to-be-processed data sets, said at least one physiological parameter of the respective one of the to-be-processed data sets, and the label of the respective one of the to-be-processed data sets,
wherein the intermediate data sets serve as the training data sets, and for each of the training data sets, the observation time interval and the total dosage of the respective one of the intermediate data sets serve as said at least one of the medication parameter of the training dataset, said at least one physiological parameter of the respective one of the intermediate data sets serves as said at least one physiological parameter of the training data set, and the label of the respective one of the intermediate data sets serves as the label of the training data set.

15. The method as claimed in claim 11, further comprising, prior to the step of obtaining a plurality of original candidate models and a plurality of original model-evaluation values, a step of:
for each of the intermediate data sets, determining whether the intermediate data set is missing a value of any one of the characteristic parameters, and when it is determined that the intermediate data set is missing any one of the characteristic parameters, deleting the intermediate data set,
wherein those of the intermediate data sets that remain serve as the training data sets.

16. The method as claimed in claim 11, further comprising, prior to the step of obtaining a plurality of original candidate models and a plurality of original model-evaluation values, a step, to be performed with respect to each of the training data sets, of:
performing standardization on any of the characteristic parameters of the training data set that is a continuous variable.
